(19) United States
(12) Patent Application Publication
(10) Pub. No.: US 2004/0034333 A1
(43) Pub. Date: Feb. 19, 2004

(54) Dialysis catheters with optimized user-friendly connections

(76) Inventors: Timothy M. Seese, South Chicago Heights, IL (US); Scott Ruddell, Gurnee, IL (US)

Correspondence Address: BAXTER HEALTHCARE CORPORATION, DF3-3E, One Baxter Parkway, Deerfield, IL, 60015, US

(21) Appl. No.: 10/222,968

(22) Filed: Aug. 19, 2002

(51) Int. Cl. A61M 25/00

(52) U.S. Cl. 604/523; 604/43

(57) ABSTRACT

A catheter is provided which has been optimized so as to become mistake-proof, user-friendly, and manually connectable to a transfer device by which the patient may hook up the catheter to a source of dialysis fluid. The catheter of the invention preferably has an outer segment which is softer than the harder internal segment and affords greater comfort to the patient. In addition, the catheter has an enlarged outer portion to lower interference and allow the catheter to be manually connected to a suitable connection device, and contains overmolded segments that can have different diameters and cross-sections so that the catheter may be optimized to meet differing requirements in any specific peritoneal dialysis operation while maintaining the integrity and structure of a non-segmented catheter. The catheter can also be made with at least two lumens wherein the lumens have a unique geometric shape so as to fit in lock-and-key fashion to suitable connection devices.

### FIELD OF THE INVENTION

The present invention relates in general to catheters used in peritoneal dialysis, and more particularly to catheters that have been optimized to allow a user-friendly means of manually connecting the catheter in a mistake-proof fashion to a device which provides access to a source of dialysis fluid while at the same time allowing for a secure and leak-free connection, and in addition maximizing the comfort of a patient having the implanted catheter.

### BACKGROUND OF THE INVENTION

Dialysis is a common treatment used to assist patients suffering from a wide variety of kidney problems including severe late stage renal insufficiency which usually results in total or near total kidney failure. This treatment cleans the blood and removes waste products and excess water from the body, a task normally performed by healthy kidneys. Presently, there are two main types of dialysis that are performed to compensate for kidney failure, namely hemodialysis and peritoneal dialysis.

In hemodialysis, the patient's blood is generally passed through a form of artificial kidney in order to cleanse it, followed by the return of the blood to the patient's bloodstream. In peritoneal dialysis, the patient's own peritoneum is used as a semi-permeable membrane in order to remove waste products, and this procedure is carried out by filling the peritoneal cavity with a dialysis solution, also known as dialysate, which preferably is introduced into the body via a permanently implanted catheter. In this process, waste products such as urea and creatinine, as well as excess water, pass from the blood through the peritoneum into the peritoneal dialysis solution, and after a given period of time, or dwell, the used dialysis solution or spent dialysate may be removed from the peritoneal cavity and then either discarded or purified for reuse. In general, the removal of the spent dialysate may be accomplished from a permanent catheter implanted in the body.

Due to the importance of the catheters in carrying out dialysis procedures, it is vitally important that the multiple connections involved in such processes be secure, leak-proof, properly aligned, and easily connectable. This is particularly important using certain catheters which need to be manually connected following implantation into a patient. In addition, since many of these devices are intended for use by a patient on an outpatient basis such as in the home without the supervision of a healthcare professional, it is important that the connections remain secure and in proper position so that there is no leakage or improper delivery of dialysis fluid. It is thus important that such connections be made as simple and effectively as possible so that secure and mistake-proof connections can readily be made manually following implantation of the catheter into a patient. Accordingly, there is a strong need to develop secure, user-friendly catheter connections and adapters which can be utilized with dialysis equipment including those units designed for home care on an outpatient basis.

Traditionally, dialysis systems have used catheters with a single lumen wherein the dialysis fluid is introduced into and removed from the patient via the same tube. However, more recently, many dialysis systems now employ multiple lumen catheters wherein it is possible to introduce dialysis fluid through one of the lumens and remove the spent dialysate through another lumen in the same catheter. An example of such a multiple lumen system is disclosed in WO 02/30489, incorporated herein by reference, wherein a dual lumen catheter is disclosed in which dialysate flows through a first lumen into the patient and spent dialysate simultaneously flows out through a second lumen. Other multiple lumen catheters have been disclosed for example in patent references including U.S. Pat. Nos. 4,950,259; 5,053,023; 5,167,623; 5,961,485; 5,868,717; 5,683,640; 5,486,159; 5,480,380; 5,380,276; 5,188,593; 5,156,592; 5,057,073; 5,009,636; 5,976,103; 5,961,486; 5,827,237; 5,569,182; 5,221,255; 4,842,582; 4,623,327; and 5,346,471; PCT references WO 94/05363 and 96/29111; European Patent Application 333308; German Patent application 10042067; and Japanese Patent references 8-206216; 2-116380 and 1-303159; all of these patent references incorporated herein by reference.

Although these multiple lumen catheters may increase the efficiency of the dialysis process, they create additional problems with regard to the multiple connections between the dialysis equipment, the lumens of the catheter, and the patient. With such multiple lumen systems, the need to create leak-proof and secure connections is even greater, and it is also important to make the systems mistake-proof and user-friendly so that the proper connections can be made easily and without confusion.

Accordingly, although numerous catheter connection systems are known, few of these even address the particular problems associated with multiple lumen catheter systems, and none provide solutions for obtaining user-friendly, secure, leak-proof connections for multiple-lumen catheters such as those utilized in peritoneal dialysis systems. Examples of such prior systems include those disclosed in patent references including U.S. Pat. Nos. 6,113,572; 4,929,236; 5,743,873; 5,129,891; 5,632,729; 5,399,165; 4,950,255; 5,417,672; 6,190,349; 5,667,490; D431,651; 5,190,529; 5,531,723; 4,781,185; 5,456,676; D303712; 6,254,589; and 6,190,372; Japanese patent references 2000-0045999; 1171499; and 8-269224; and European Patent EP 553254 B1; all of these patent references incorporated herein by reference,

Another problem with catheters that are implanted in association with peritoneal dialysis systems is that some of these catheters, such as the dual lumen catheter disclosed in WO 02/30489, have an implanted section which is made of a relatively hard material such as hard silicone to maintain a proper configuration inside the peritoneal cavity when implanted. However, if such harder material is utilized in the part of the catheter that will be located externally, it can create some discomfort for a patient. Although certain patents relate to catheters having a transitional area with an intermediate durometer, such as U.S. Pat. Nos. 6,135,992; 5,451,206; 5,348,536; 5,125,913; 5,792,124; and 4,739,768, all incorporated herein by reference, none have provided the necessary means whereby a dual-lumen catheter system can be simply and effectively made without such complex and hard to manufacture materials, and yet provide the necessary internal durometer while maximizing comfort to the patient, being manually connectable, and providing a secure and leak-proof connection.

Further, other prior catheters utilized in procedures such as Continuous Flow Peritoneal Dialysis (CFPD) have had lumens which have a high interference with catheter connection ports used to connect the patient to a source of dialysis fluid, and this makes connection even more difficult to accomplish.

Therefore, in addition to providing a connection assembly for multiple lumen catheters which is user-friendly and mistake-proof, so as to prevent the chance of an erroneous connection, it would be beneficial if the catheter used in connection with this assembly was more comfortable for the patient and could be connected easily with a minimum of resistance or interference.

Finally, in addition to providing a system of connections for multi-lumen catheters which can be made user-friendly and mistake-proof, it is also desirable to develop a system wherein the patient can perform dialysis in the home setting and thus stop or initiate the flow of dialysis fluid into or from the patient's peritoneal cavity, such as during the start or end of a dialysis procedure. Previously, systems wherein the patient could initiate or halt a dialysis operation involving a single-lumen catheter have been known, such as the MiniCap 6 month transfer set produced by Baxter Healthcare Corporation as described in the MiniCap brochure, incorporated herein by reference. However, because this system was specifically designed for use with single lumen catheters, it was not sized or shaped to conduct dialysis fluid from a multiple-lumen catheter, nor did it possess any means for ensuring the proper orientation as would be crucial for any devices transferring fluids from a dual-lumen catheter where fluid may be going into and out of the patient at the same time.

Accordingly, it is also desirable to develop a user-friendly transfer set for a multiple lumen catheter by which the user may easily and effectively set up the catheter tubing for dialysis and initiate or stop the flow of dialysis fluid when necessary during a dialysis operation involving a catheter having at least two lumens, i.e., a catheter which will allow both inflow and outflow at the same time.

Therefore, it is important and highly desirable to develop new catheters, catheter connection adapters and patient transfer sets which are user-friendly, mistake proof and which can provide secure and leak-proof connections for systems utilizing multiple lumen catheters, particularly for implanted multiple lumen catheters such as are employed in peritoneal dialysis systems

### SUMMARY OF THE INVENTION

It is thus an object of the present invention to provide improved single and multiple lumen catheters which provide ease of use and which maximize comfort to the patient.

It is further an object of the present invention to provide catheters which have internal segments with the appropriate high durometer for use in a peritoneal dialysis operation yet which also have external segments of lower durometer which will be softer than the internal segment, easy to connect, and which will allow for a more comfortable fit for the patient.

It is still another object of the present invention to provide catheters with an enlarged outer end so as to reduce the interference between the lumens of the catheter and the connector ports which are used in connecting the patient to a source of dialysis fluid and make it easier to manually insert the connector ports into the end of the catheter, all while maintaining the smaller diameter of the catheter at its exit point from a patient.

It is yet another object of the present invention to provide a catheter which has been segmented into different regions with different properties which allows for the catheter tubing to be optimized to meet differing requirements of the catheter when employed inside the peritoneal cavity.

It is another object of the present invention to provide a catheter that can be manufactured from segments having different properties, such as different durometers, different inner and/or outer diameters, and different shapes, and yet which maintains the integrity and structure of a unitary catheter.

It is even further an object of the present invention to provide a multiple lumen catheter having at least two lumens in which the geometries of an upper and lower lumen differ in a particular manner so as to ensure proper insertion of connecting devices having upper and lower profiles which match the particular geometry of the upper and lower catheter lumens in lock-and-key fashion.

These and other objects are provided by virtue of the present invention which comprises a single or multiple lumen catheter which has been optimized for user-friendly connections and maximum flexibility by virtue of features which improve the comfort of the wearer of the implanted catheter, namely a patient undergoing peritoneal dialysis procedures, and which assist in the ability of the catheter to be correctly connected to an adapter and/or other transfer equipment which connects the catheter to a source of dialysis fluid. In particular, these features include the provision of a segmented catheter having a first segment of a relatively hard durometer which is designed to be implanted into a patient and a second segment designed to come out of the patient and be connectable to other dialysis equipment which has a relatively soft durometer which will thus increase comfort to the patient. In other embodiments of the present invention, a catheter having an enlarged external end is provided so as to minimize interference when it is necessary to manually connect the catheter of the invention to connectors which will connect it to dialysis equipment such as for the initiation of a dialysis procedure. Even further, it is possible to construct the catheter of the present invention so as to be segmented wherein the segments have different diameters and shapes, and this is accomplished, for example, by overmolding the various segments so as to constitute a single catheter, even where the catheter will have at least two lumens, yet which still operate with the integrity and structure of a unified catheter. This embodiment is particularly advantageous under those circumstances wherein it may be necessary to have the catheter tubing optimized to meet different requirements of the catheter inside the peritoneal cavity, subcutaneously, and extracorporeally. In addition, multiple lumen catheters are provided which have different geometries for at least an inflow and an outflow lumen, such as catheter having an upper and lower lumen, which are designed to fit in mistake-proof lock-and-key fashion with mating connection adapters which are constructed so as to have upper and lower ports with matching profiles to the upper and lower lumens of the catheter of the present invention.

These embodiments and other alternatives and modifications within the spirit and scope of the disclosed invention are described in, or will become readily apparent from, the detailed description of the preferred embodiments provided herein below.

### BRIEF DESCRIPTION OF THE DRAWING FIGURES

FIG. 1 is a perspective view of a catheter in accordance with the present invention having an external segment with a lower durometer than a harder internal segment designed to be implanted into a dialysis patient.

FIG. 2 is a schematic view showing the catheter of FIG. 1 as implanted in a dialysis patient.

FIG. 3 is a perspective view of an alternate embodiment of the catheter of the present invention wherein the external end is enlarged to provide easier connections with other dialysis equipment.

FIGS. 4A, 4B and 4C are cross-sectional view of the catheter of FIG. 3 at lines C-C, B-B, and A-A, respectively.

FIGS. 5A, 5B and 5C are cross-sectional view of an alternative design of the catheter of FIG. 3 showing the alternate cross-section at the locations indicated at lines C-C, B-B, and A-A, respectively, of FIG. 3.

FIG. 6 is a side, cutaway view showing the inner structure of an alternative embodiment of the catheter of the invention illustrating the overmolding between an internal segment and an external segment having different inner and outer diameters.

FIG. 7 is a perspective view of an alternative embodiment of a multiple lumen catheter of the present invention alongside a connection adapter having ports of matching size and shape.

FIG. 8 is a perspective view of an alternative embodiment of a multiple lumen catheter in accordance with the invention alongside a matching connection adapter.

FIG. 9A is a cross-sectional view of a multiple lumen catheter in accordance with the invention that has been designed to match the configuration of the ports of a suitable connection adapter.

FIG. 9B is a cross-sectional view of an alternative multiple lumen catheter in accordance with the invention that has been designed to match the configuration of the ports of a suitable connection adapter.

FIG. 9C is a cross-sectional view of an alternative multiple lumen catheter in accordance with the invention that has been designed to match the configuration of the ports of a suitable connection adapter.

FIG. 9D is a cross-sectional view of an alternative multiple lumen catheter in accordance with the invention that has been designed to match the configuration of the ports of a suitable connection adapter.

### DETAILED DESCRIPTION OF THE PREFERRED EMBODIMENTS

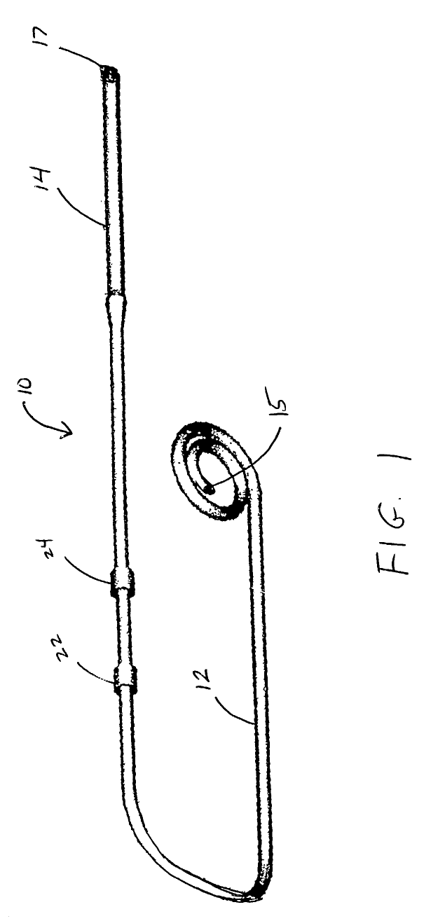

As shown in the accompanying drawing figures, in accordance with the present invention, there is provided a catheter for use in a peritoneal dialysis system which has been constructed so as to be easily connected to dialysis equipment such as a catheter connector which connects the catheter to a source of dialysis fluid, and/or to a source to receive spent dialysate, and which exhibits improved comfort to the patient over prior catheters made of a uniform hard silicone material. In one preferred embodiment in which patient comfort is maximized, the catheter of the invention comprises catheter tubing in at least two different segments, each having a uniform tubing durometer (a measure of stiffness/softness), but wherein the durometers differ between segments so that there are softer segment and harder segments. In the preferred embodiment, as shown in FIG. 1, the catheter 10., which may be a single lumen or multiple lumen catheter, is formed from at least two segments, including an internal harder segment 12 which has an end 15 which is implanted into the patient, and segment 12 will reside in the peritoneal cavity and possibly subcutaneously as well. There is also provided an external softer segment 14 having an end 17 which will be external to the patient, and this section is either fully or partially external to the patient and which has a lower durometer than internal implanted segment 12. The external segment 14 will preferably extend from the proximal (or external) end 17 of the catheter 10 to a point 18 just slightly outside of the exit side 19, as shown for example in FIG. 2. The external softer segment may also extend subcutaneously in the patient as well.

In the preferred embodiment, the internal segment 12 will thus be of a higher durometer than external softer segment 14 and will thus be made of a harder material. Although one skilled in the art will understand a variety of durometers for the external segment 14 will afford ease and comfort to the patient, and a variety of durometers will enable the implanted segment 12 to have suitable durability and strength to carry out the dialysis procedure inside the patient, it is generally preferred that the softer segment 14 have a durometer in the range of about 45-55, and the internal segment 12 have a durometer in the range of about 55-75. In the particularly preferred mode, external segment 14 has a durometer at or around 50 and the internal segment 12 has a durometer at or around 65. In this embodiment, the external softer segment is extruded and formed with the lower durometer will provide greater comfort to the patient and will also improve the ease of use and sealing of the connection at the external end of the catheter which will be hooked up through connection means to a peritoneal dialysis device. The internal segment of higher durometer will be a harder material which will be suitable for maintaining the necessary formed configuration inside the peritoneal cavity. When implanted, the internal segment will thus reside inside the peritoneal cavity, and the transition from a hard durometer to the softer durometer is preferably made at a point near the exit point of the catheter emerging from the patient after the catheter is implanted. The preferred material for the catheter of the invention will be silicone, but other suitable materials which are flexible, medical grade, fluid-tight tubing and which can be obtained at the particular durometer required for the particular segment as set forth above may be utilized in the invention. The present invention thus allows the catheter tubing durometer to be optimized to meet differing requirements of the catheter as it will be employed not only inside the peritoneal cavity, but subcutaneously and extracorporeally as well. In the example shown in the drawing figures, a multiple lumen catheter is shown which has at least one lumen for inflow and one lumen for outflow, but it is understood that the present invention may also be utilized in single lumen catheters or multiple lumen catheters having more than two lumens.

As indicated above, in the preferred operation of the present invention, the internal segment 12 of catheter 10 is designed to be implanted into the patient, as best shown in FIG. 2. As observed in this drawing figure, the catheter 10 of the present invention has its internal end 12 implanted in the patient 20 so that the internal end 15 and the remainder of this tubing segment will reside in the peritoneal cavity so as to be useful in a dialysis operation. The external softer segment 14 is positioned outside patient 20, and generally will extend from external end 17 to a point 18 outside of patient entry site 19. If so desired, the softer external segment may also extend to a point just below the exit point of the catheter from the patient. In the preferred embodiment, the catheter of the invention may also contain cuffs 22 and 24 respectively to assist in stationing the catheter 10 in patient 20. For example, these cuffs, which may be made of a suitable physiologically compatible material such as polyester, porous silicone, or other materials which will allow tissue ingrowth, may be positioned so that cuff 22 which is closer to the internal end 15 of the catheter 10 can be imbedded in muscle tissue. In this embodiment, cuff 24 is positioned just below the patient's skin and just prior to entry site 19. These cuffs will allow subcutaneous tissue to grow inside of them thus further anchoring the catheter 10.

Once implanted in this fashion, the external end 17 of catheter 10 may then be utilized to initiate a peritoneal dialysis procedure by being connected to a source of peritoneal dialysis fluid via a suitable connection device or transfer set. Such connection assemblies are disclosed for example in co-pending U.S. patent application Ser. No. ______ of Seese et al., and transfer sets suitable for use with the catheters of the present invention are disclosed for example in copending U.S. patent application Ser. No. ______ of Seese et al., both applications being incorporated herein by reference. In general, any suitable connection device whereby catheters such as the catheters of the invention may be connected so as to be utilized to transfer dialysis fluid into and out of a patient may be employed in accordance with the present invention.

In another embodiment of the present invention designed to allow for efficient attachment of the catheter to a transfer set so that it can be connected to a source of dialysis fluid, a catheter is provided which has an enlarged external end so as to make it easier for the catheter to be connected to a catheter adapter. In some cases involving present catheters including dual lumen catheters, connections are usually made by simultaneously inserting dual connector ports into the separate lumens of the catheter. However, in certain cases, these connector ports may be larger than the catheter lumens and thus require the catheter to be stretched around the connector end for insertion. The large connector ports are generally required to accommodate required connector flow rates and moldable wall thicknesses, but at the same time, a generally smaller internal catheter is usually required to reduce the size of the catheter exit site so as to enhance healing of the site and reduce the potential for exit site infections. This results in a generally high interference between the smaller sized catheter end and the larger sized connector ports which renders manual insertion of the connector into the catheter somewhat difficult.

In accordance with the present invention, a catheter with an enlarged external end segment is provided which mates with the connector and allows for enhanced ease of connection to the mating connectors. The enhanced ease of connection afforded by the present invention allows for stronger, more integral manual connections to be made easily and securely after the catheter is implanted in the body of the patient. Because this type of catheter cannot be connected during manufacture but instead must be connected after implantation, it is important to make this connection simple to accomplish manually, yet leak-proof, secure and mistake-proof as well, and the enlarged external end helps for all of these purposes. In accordance with the invention, while the external end of the catheter is enlarged, the internal segment may remain the same size and may thus be optimized at a smaller diameter so as to reduce the size of the catheter exit site and lower the risk of exit site infections.

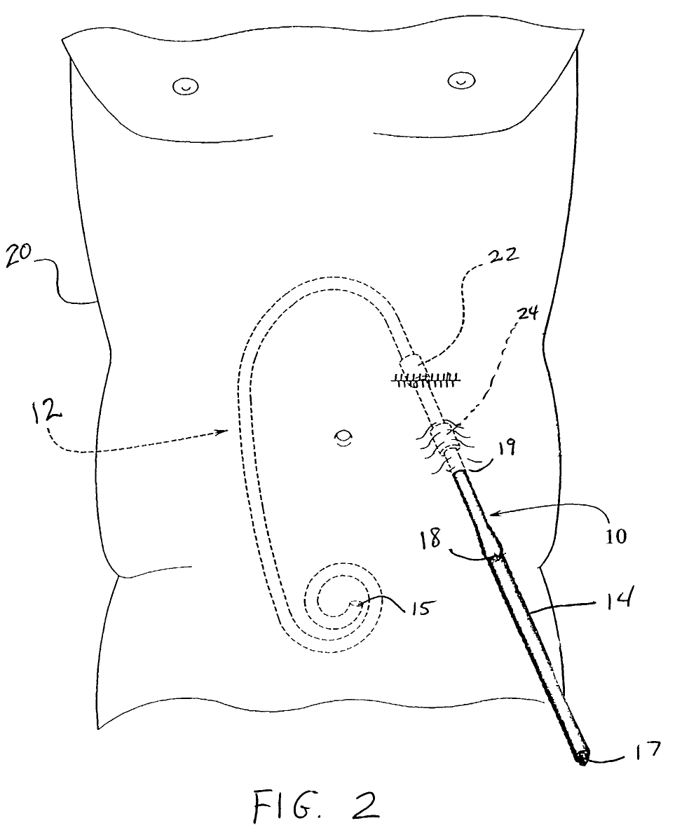

As shown for example in FIG. 3, a catheter 30 in accordance with the present invention comprises an internal segment of catheter tubing 32 which is the segment including the internal end 25 of catheter 30 and is designed to be implanted in the patient. This segment 32 will have a normally-sized lumen configuration and diameter. The catheter 30 has at its other end the external segment 34 which will have an enlarged diameter at the end 37 of the catheter which is designed to remain external from the patient. Once again, although this embodiment is shown with regard to a dual-lumen embodiment, the invention may be employed in catheters having a single lumen or more than two lumens. In the present embodiment, the first segment may be attached to the second segment in any suitable way known in the art, for example by butt welding the ends that meet at point 33, which in this case is below cuffs 36 and 38 which are designed to be implanted in the patient. It is also possible that the attachment point can be made adjacent to or even underneath the cuffs on the catheter. Still other methods of making the attachment between the internal and external segments are possible, such as utilizing the overmolding process described further below. Even further, an alternative method of joining an internal and external segment of the catheter of the invention would be to use a third segment which is positioned between the internal segment and the external segment, and which will have a similar internal geometry to the internal and external segments at the point of attachment. This connector segment of the catheter can be attached to the internal and external segments through any suitable method such as through the overmolding process described below, or via an adhesive material such as a silicone paste.

In any event, the catheter 30 is designed so that the point 39 at which the catheter 30 will emerge from the patient will have a normally small diameter so as to reduce risk of infection at the exit site. Accordingly, the external segment 34 of catheter 30 will have a smaller diameter at the end near the patient exit site 39, and will have an enlarged diameter at the proximal or external end 37 of catheter 30, and the transition from small to large diameter may be accomplished in any suitable manner and will always be located outside of the body when the catheter 30 is implanted. For example, in the embodiment shown in FIG. 3, the enlargement of the catheter 30 takes place in a gradual transition zone 35 wherein the diameter gradually increases until the final desired enlarged diameter is achieved at the end 37 of catheter 30. In this embodiment, a wide variety of diameters and geometric properties are possible in the transition between the smaller internal segment 32 and the enlarged end 37 of segment 34, with the transition between the smaller and larger segments being achieved in any of a number of suitable ways. In one preferred method, this is achieved by overmolding of the two segments, as will be described further below. Alternatively, this transition segment which goes from a small lumen diameter to a larger lumen diameter may also be prepared by extruding the catheter segment to have the geometry wherein the diameter goes from smaller to larger.

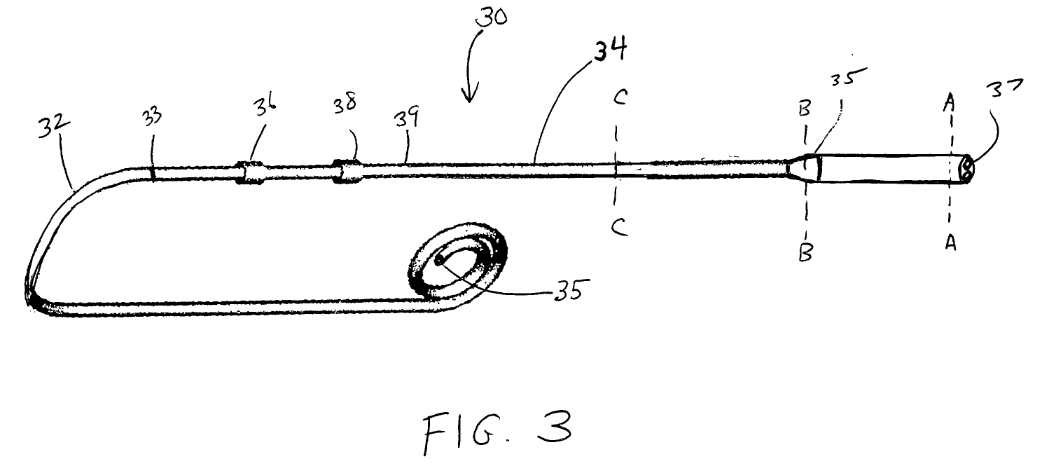

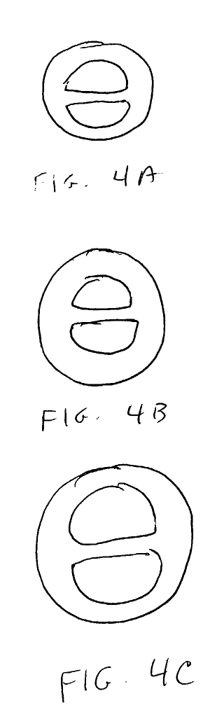

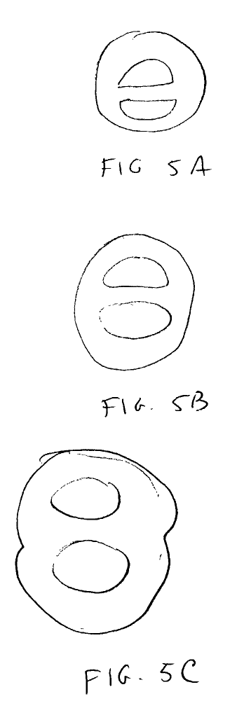

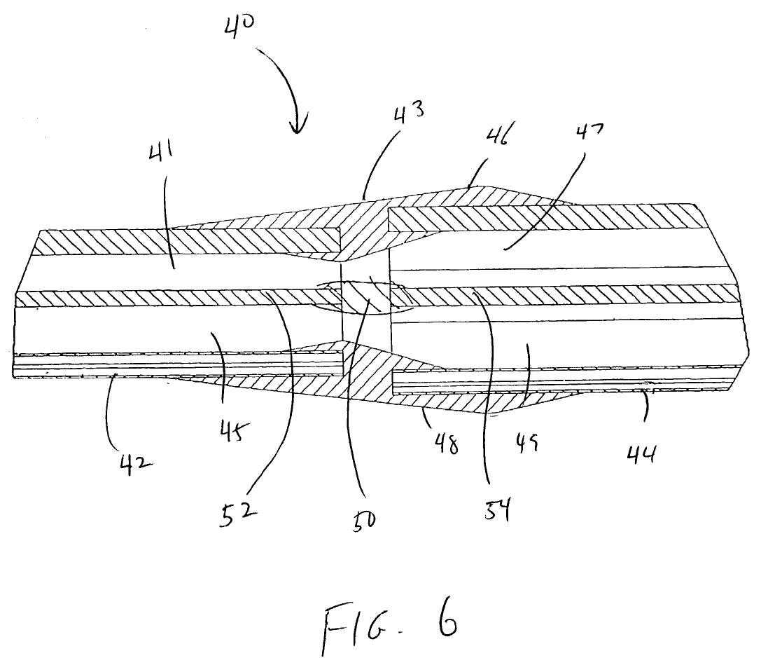

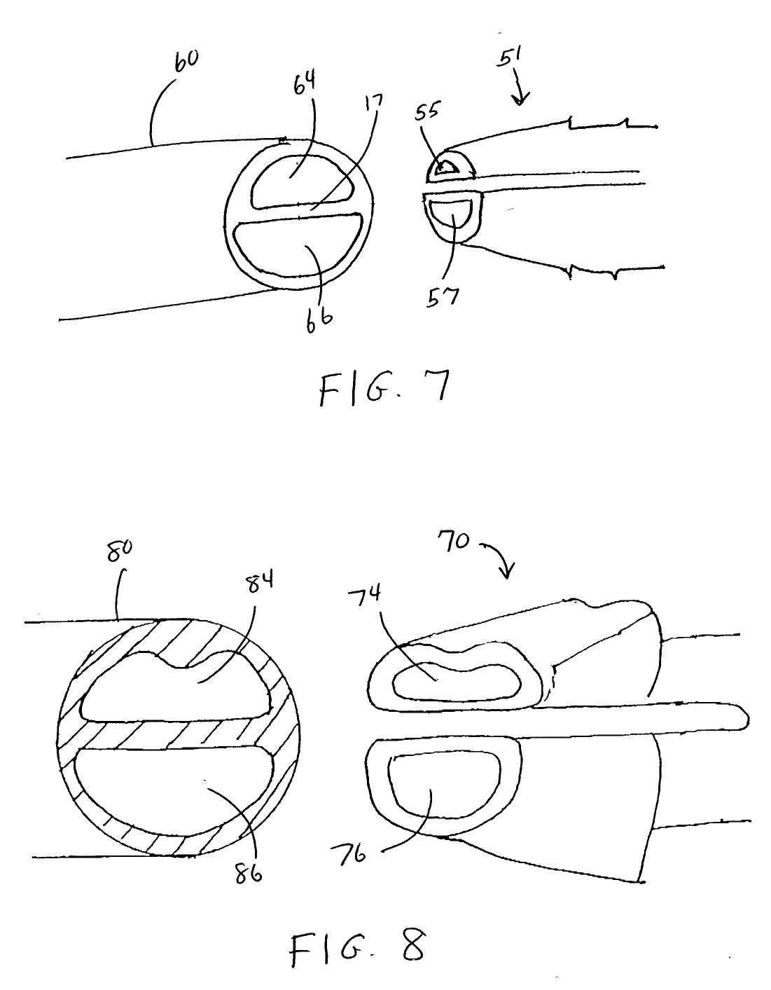

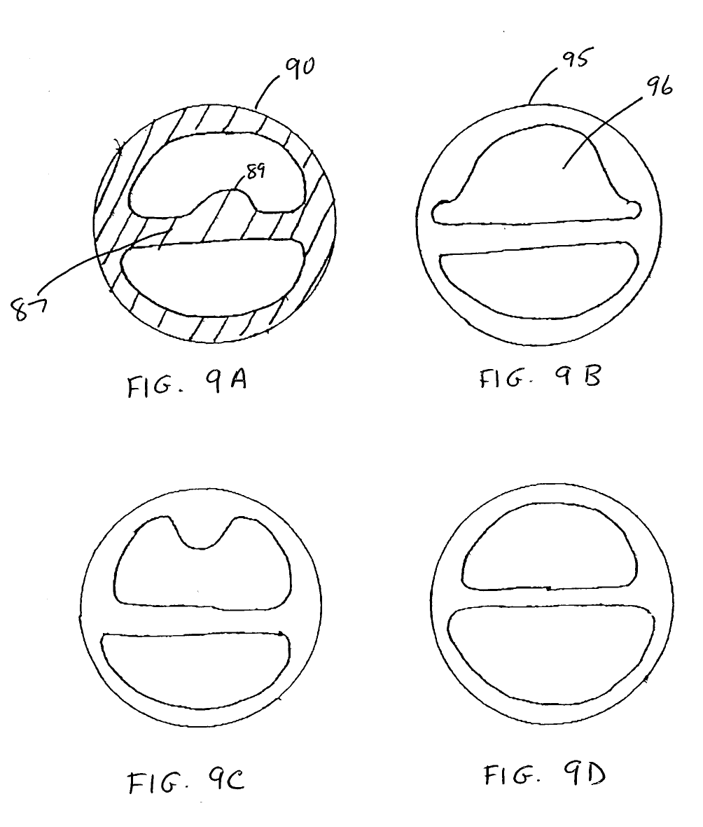

As shown in the cross-section of FIG. 4, wherein FIG. 4A is a cross section of the catheter at the lines C-C, FIG. 4B is a cross-section at lines B-B, and FIG. 4C is a cross-section at lines A-A, the outer diameter of the catheter 30 increases from the smaller diameter at the exit site, to a middle diameter in transition zone 35 to a final enlarged diameter at the end 37 which will be the end that will need to be hooked up to a fluid connection device. In FIGS. 4A-4C, one mode of the catheter of the invention is to retain the double-D shape as the catheter is enlarged. However, it is also possible to change the configuration of the lumens of the catheter of the invention such that the shape of the lumens in the segment emerging from the patient is double-D, but this can gradually change to a double oval shape at the external end 37, as shown in FIGS. 5A-5C which show the alternative cross-sections at lines C-C, B-B and A-A of FIG. 3, respectively. In accordance with the invention, the external end 37 of catheter 30 is thus enlarged so as to allow easier connection of the ports of a connection adapter into the lumens of catheter 30 and make it easier for the catheter to be manually attached to connection equipment after the catheter is implanted into a patient. As indicated above, although described with regard to a dual-lumen embodiment, a catheter having the enlarged end in accordance with the invention may also be a single lumen catheter or may have more than two lumens.

In another preferred embodiment of the present invention, there is provided an overmolded section which connects internal and external segments of the catheters of the present invention, and which makes these segments, which may be of different internal or external diameter, or even different geometries, into an integrated whole catheter. This overmolded section may be observed, for example, in FIG. 3 wherein overmolded section 35 comprises the transition zone between a segment of catheter 30 which has the same inner diameter as internal segment 32 and the external end 37 of catheter 30 which has an enlarged end and thus a greater outer diameter than the internal segment 32. The external segments which are attached to internal segments may also be of different durometer, as is described above with regard to catheter 10 and as shown in FIG. 1 wherein an external segment 14 has a different durometer than internal segment 12. In this embodiment of the invention, the external segments of the catheter may differ from internal segments in size, shape, geometry and durometer so as to provide different regions which can accomplish different functions depending on their location in the implanted catheter. In addition, the overmolding can connect two segments of the catheter of the invention, e.g., the internal and external segments, at a point wherein the diameters and geometric shape are the same, such as would be the case with the embodiment shown in FIG. 1 wherein the two segments primarily differ with regard to durometer.

This embodiment of the invention is thus an improvement over conventional peritoneal dialysis catheters which are typically constructed of a single tubing segment of uniform size. By segmenting the catheter in accordance with the invention, the present catheter may be optimized to meet the differing requirements for the catheter when situated either in the peritoneal cavity, subcutaneously and extracorporeally. In the present invention, segments having separate and distinct diameter, durometers, or other properties may be joined directly onto one another, thus allowing for segments of varying properties to be joined into unified catheters in a leak-proof manner that preserves the integrity and structure of the whole catheter.

In accordance with the invention, as observed for example in the cross-section of FIG. 6, there is provided a catheter 40 comprised of differing segments, such as internal segment 42 and external segment 44, which may differ from each other in a variety of characteristics, such as durometer, outer diameter, inner diameter, and even lumen geometry. The overmolded section 43 is designed to act as a transition zone between internal segment 42 and external segment 44, and allow a single catheter to be formed wherein a particular diameter, shape, hardness or other characteristics are featured in the internal segment, and different diameters, shape, hardness or other characteristic are featured in the other segment. As shown in FIG. 6, the overmolded section 43 includes an upper outer portion 46, which forms the outer transition between upper lumen 41 of internal segment 42 and upper lumen 47 of external segment 44. As shown in the drawing figure, this embodiment can provide an overmolded section which will link an internal segment of smaller diameter with an external segment of larger diameter. Additionally, the overmolded section 43 includes a lower outer portion 48 which provides the transitional wall between the outer portion of lower lumen 45 of internal segment 42 and lower lumen 49 of the external segment 44. Finally, overmolded section 43 also contains a central section 50 which provides a connection between the middle wall or septum 52 of internal segment 42 and the middle wall or septum 54 of external segment 44. Once again, the overmolded section 43 acts to provide a transition between internal segment 42 and external segment 44 so that fluid flow along the upper and lower lumens will be conducted as in a whole catheter without such segments.

In the preferred process to obtain the overmolded catheter in accordance with the present invention, the separate sections may be separately extruded before being connected through the overmolding process of the present invention. For example, the internal segment to be joined may be extruded and formed of high durometer (e.g., about 65) silicone material, and the external segment may be extruded and formed of silicone or other suitable material at a lower durometer, e.g., about 50. In addition, as shown in FIG. 6, the external segment may have a larger tubing outer diameter, different inner tubing diameter and different internal lumen geometries than the internal segment.

In accordance with the invention, the two segments may be bonded to each other using any suitable procedure which will provide a unitary and leak-proof transition between the differing segments, but in the preferred process, an overmolding procedure is carried out as follows. In this process, a mandrel or molding core pins are inserted through the entire length of both lumens of the external catheter segment and into the mating lumens of the internal catheter portion. The core pins fit snugly against the inner walls of both catheter segments, but leave space between the walls near the bonding region for the overmolding material, e.g., a suitable silicone or other moldable plastic, to flow into and bond with each segment. The segments are brought together with a spacing of specified length to allow for additional overmold material bonding. A mold cavity is preferably placed around the bonding region which will form the outer geometry of the overmold material joining the two segments. A suitable catheter bonding material is then injected into the mold which fills all spaces within the catheter overmold cavity, between the tubing segments and core pins, and between the ends of the tubing segments to be bonded. This forms a third overmolded component which acts to bond the two separate catheter segments forming an integral, whole catheter having an internal segment that can have one diameter and durometer, and a second external segment that may have a softer durometer and greater diameter.

Alternatively, the above catheter can be constructed by overmolding the internal catheter segment rather than by overmolding a second pre-extruded segment. In this case, a mold cavity for the geometry of the external catheter segment would be placed at the end of the internal catheter segments and core pins would traverse the length of the overmold cavity and enter the end of the internal catheter segment. The external catheter segment would be formed by molding onto the internal segment with these cores and cavity. Still other alternatives to the above mode are possible, including having the bonding region or transition zone varying in location along the catheter length, depending on the catheter requirements. Beyond the mold cavities discussed above, reinforcement materials and other similar structures may be used inside or outside of the overmolded regions to help enhance the strength and integrity of the overall catheter in these zones. The specific geometries of the internal and external segments in accordance with the invention may thus vary greatly, and need not be uniform throughout if so desired. In addition, segments of different durometers may be interspersed as necessary to provide catheters to meet specific patient needs yet at the same time maximize comfort to the patient. Although the overmolding process has been is described herein with regard to a multiple-lumen embodiment such as a catheter having at least two lumens, it may also be utilized for single lumen catheters as well.

In another embodiment of the present invention, a multiple-lumen catheter is provided wherein the ends of the catheter will be mated with a catheter adapter in lock-and-key fashion to ensure a mistake-proof connection is provided so that new dialysis solution will be directed into the patient and spent dialysate will be channeled out of the patient. In prior multiple lumen catheters, one embodiment had the internal lumens having a back-to-back “Double-D” shape wherein the internal geometry was the same right-side-up as well as upside-down. However, in accordance with the present invention, it is possible to provide catheters having at least two lumens, e.g., one for inflow and one for outflow, wherein the middle wall or septum 17 is offset from the center, as shown in FIG. 7, which results in an asymmetric double-D pattern wherein one of the ports is smaller and one of the ports is larger. As shown in FIG. 7, this catheter is designed to mate with a connection adapter 51 that has a smaller upper port 55 and a larger lower port 57 which fit, respectively, into upper lumen 64 and lower lumen 66, of the catheter 60, respectively. In accordance with the invention, the asymmetrical pattern of cross-sections for the upper and lower lumens of the catheter of the invention provides a means whereby the proper positioning and alignment of the catheter and a mating connector can take place because with a different upper and lower section, the adapter ports will only fit one way into the dual lumen catheter used in accordance with the invention. Accordingly, the asymmetric nature of this embodiment will provide a user-friendly means of indicating the proper configuration for connecting the catheter to the adapter in accordance with the invention.

In a further embodiment in accordance with the present invention, the catheter may be designed with its upper and lower lumens having a specific geometric shape that is keyed to the matching shape of a connection adapter which will have ports sized and shaped to allow the connection in unique lock-and-key fashion. In this manner, once again the catheter and connector are made mistake-proof since the ports of the adapter can only be placed into the dual lumen catheter in the specific manner wherein the outer shape of the ports matches the internal cross-section of the lumens of the catheter, and this ensures proper alignment and positioning of the adapter and catheter so that a safe and mistake-proof peritoneal dialysis procedure can take place. As shown in FIGS. 8 and 9A-9D, this embodiment can take on a variety of different lumen shapes, in each case the shape of the lumens corresponding to matching shapes in the connection adapters so as to receive the port of the unique geometrical configuration in such a position which ensures proper alignment and functioning of the peritoneal dialysis system.

For example, as shown in FIG. 8, an adapter 70 has a lower port 76 having the D-shaped as shown in embodiments described above, but wherein the upper port 74 has a unique configuration featuring a generally U-shaped depression or notch at the top central portion of the upper port. In accordance with the invention, this particular shape of the ports will be matched in the multiple lumen catheter 80 of the present invention wherein the internal cross-section of the upper lumen 84 has the same shape as the outer surface of the upper port 74 so as to allow the adapter to be inserted properly in the catheter. Similarly, as also shown in FIG. 8, the lower lumen 86 of catheter 80 is sized to match with the lower port 76 of adapter 70. As shown for example in the drawing FIGS. 9A-9D, numerous alternative designs are possible, including a configuration wherein the upper lumen of the catheter of the invention has a notch or projection 89 on the upper surface of the septum or middle wall 87 of catheter 90, as shown in FIG. 9A. Alternatively, a catheter 95 is shown in FIG. 9B which has a roughly bell-shaped upper lumen 96 which will match up with a similarly-sized and shaped upper port of an adapter (not shown) used to connect the catheter 95 in accordance with the invention. Still other designs for the cross-section of the lumens of multiple-lumen catheters of the invention which will match up with similarly sized and shaped ports of suitable connection adapters are shown in FIGS. 9C and 9D.

In the preferred operation, the catheters as described above are implanted into the patient whereby the internal segment which carries out the dialysis procedure is situated and maintained in the patient's peritoneal cavity, such as through porous cuffs which may be sutured to muscle tissue or implanted subcutaneously, and the external segment which emerges from the patient will be able to be connected to a source of dialysis fluid and/or a means for receiving spent dialysate following the dialysis procedure. The connection means may be a suitable catheter connector or other transfer set capable of conducting fluid from the catheter into and out of the patient as appropriate, and the connection may be made manually following the implantation of the catheter into the patient. In the preferred mode of operation, the external end of the catheter will be hooked up to a permanent transfer set assembly which can be capped when not in use, and thus when it is desired to initiate a peritoneal dialysis operation, the patient removes the cap, hooks the catheter up to the source of dialysis fluid, undoes any clamping means around the tubing, and allows a dialysis procedure to proceed. Following the completion of the dialysis procedure using the catheters of the present invention, the catheters may be clamped off, and the connection to the source of dialysis fluid is disengaged, after which the transfer set is recapped until the next procedure will take place.

In summary, through the use of the catheters of the present invention, a safe and effective dialysis procedure can be carried out, such as a continuous flow peritoneal dialysis procedure which can be carried out by the patient in the home setting, and the present invention will ensure that the catheter is hooked up properly so as to ensure secure, accurate and leak-proof flow between a patient and a supply of dialysis fluid as utilized during the dialysis procedure.

The present invention has been described above with regard to exemplary embodiments, but as will be understood by those of ordinary skill in the art, the present invention encompasses numerous additional embodiments which will fall within its scope in addition to the specific embodiments described above.

What is claimed is:

1. A catheter capable of being used in a peritoneal dialysis system comprising catheter tubing segmented into at least two regions each having a uniform durometer, and wherein the durometer in one segment is different than the other.

2. A catheter according to claim 1 wherein there is at least one segment which will be implanted into the patient and one segment that will be at least partially external to the patient, said implanted segment having a harder durometer than the segment that is at least partially external to the patient.

3. A catheter according to claim 2 wherein the external segment has a durometer in the range of about 45-55 durometers.

4. A catheter according to claim 2 wherein the external segment has a durometer of about 50.

5. A catheter according to claim 2 wherein the implanted segment has a durometer in the range of about 55-75 durometers.

6. A catheter according to claim 2 wherein the durometer of the implanted segment is about 65 durometers.

7. A catheter according to claim 1 having at least two-lumens.

8. A catheter for use in a peritoneal dialysis system which comprises a catheter having an internal end that is to be implanted in the patient and an external end which allows the catheter to be connected to a source of dialysis fluid, wherein the diameter of said external end is greater than the diameter of said internal end.

9. A catheter according to claim 8 wherein the catheter is segmented into two portions, an internal portion containing the internal end of the catheter and an external segment containing the enlarged external end of the catheter.

10. A catheter according to claim 8 wherein the catheter is uniform in diameter until at least a point on the catheter which will be located external to the patient after the catheter is implanted into a patient.

11. A catheter according to claim 8 having at least two lumens.

12. A catheter according to claim 11 wherein the geometric shape of at least two lumens at the beginning of the internal segment is a double-D pattern, and the geometric shape of the two lumens at the enlarged end of the external segment is a double oval.

13. A catheter for use in a peritoneal dialysis system which comprises a catheter having an internal segment having a given diameter and an external segment having a different diameter and an overmolded section between the internal and external segments which joins these segments and forms an integral catheter with a leak-proof transition between internal and external segments.

14. A catheter according to claim 13 wherein the internal segment has a different durometer than the external segment.

15. A dual lumen catheter according to claim 13 wherein the internal segment has a different internal diameter than the external segment.

16. A dual lumen catheter according to claim 13 wherein the internal segment has a different external diameter than the external segment.

17. A dual lumen catheter according to claim 13 wherein the internal segment has a different internal geometry than the external segment.

18. A dual lumen catheter according to claim 13 wherein the internal segment has the same diameter than the external segment at the point where the segments are attached.

19. A dual lumen catheter according to claim 13 wherein the internal and external segments are formed using separate extrusion processes.

20. A dual lumen catheter according to claim 13 further comprising additional segments which are overmolded to form a whole integral catheter.

21. A dual lumen catheter according to claim 13 wherein the overmolded section is formed by inserting pins through the entire length of the catheter lumen of one of the segments and into the mating lumen of the other segment so that the pins fit snugly against the inner walls of both segments but leave space between the walls near the bonding area, surrounding the area where the segments are to be bound with a mold cavity, and injecting a catheter bonding material into the mold cavity which fills the spaces within the mold cavity and between the tubing segments and the pins and between the ends of the segments, and allowing the bonding material to set to form the overmolded section and bond the internal and external segments.

22. A catheter according to claim 13 in combination with a catheter connection assembly capable of transmitting fluid from the catheter to a peritoneal dialysis fluid source and vice versa.

23. A catheter according to claim 13 having at least two lumens.

24. A method of making the catheter according to claim 13 comprising inserting pins through the entire length of the catheter lumen of either the internal or external segment of catheter and into the mating lumen of the other segment so that the pins fit snugly against the inner walls of both segments but leave space between the walls near the bonding area, surrounding the area where the segments are to be bound with a mold cavity, and injecting a catheter bonding material into the mold cavity which fills the spaces within the mold cavity and between the tubing segments and the pins and between the ends of the segments, and allowing the bonding material to set to form the overmolded section and bond the internal and external segments.

25. A multiple lumen catheter suitable for use in a peritoneal dialysis system comprising catheter tubing having at least two lumens, wherein the configuration of the lumens of said tubing has a keyed geometric shape that will match with a mating geometric shape of the ports of a connection device that can be inserted into said lumens so that the connection between said catheter and said connection device can only be made when the ports and the lumens are correctly aligned.

26. A multiple lumen catheter according to claim 25 wherein the catheter has at least one upper lumen and one lower lumen.

27. A multiple lumen catheter according to claim 26 wherein the catheter a projection on the lower surface of its upper lumen which is designed to mate in proper alignment with a connection device having a notch on the lower surface of the upper port of said connection device.

28. A multiple lumen catheter according to claim 26 wherein the upper lumen of the catheter has a U-shaped notch at the top surface of the upper lumen which is designed to mate in proper alignment with a matching notch on the upper port of a connection device.

29. A multiple lumen catheter according to claim 26 wherein the middle wall of the catheter is offset so as to create an upper and lower lumen wherein one lumen is greater in cross-section than the other.
